(19) United States
(12) Patent Application Publication
(10) Pub. No.: US 2007/0255734 A1
(43) Pub. Date: Nov. 1, 2007

(54) IMAGE READING SYSTEM

(76) Inventor: Katsushi MORIMOTO, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/733,425

(22) Filed: Apr. 10, 2007

(30) Foreign Application Priority Data
Apr. 17, 2006 (JP) 2006-113288
Apr. 2, 2007 (JP) 2007-096755

(51) Int. Cl. G06F 17/00; A61B 5/05; G06F 7/00

(52) U.S. Cl. 707/100

(57) ABSTRACT

A system obtains image data from reading a document by means of a scanning function; creates positional information indicating an area of a particular article included in the image data; creates meta data including bibliographic information of the article, information identifying the article and the positional information; stores the thus-created meta data; delivers to a terminal at least the bibliographic information and the information identifying the article; delivers to the terminal the image data of the article in response to a request; and carries out display control of the article image based on the positional information when the meta data has the positional information of the article, while, when the meta data does not have the positional information of the article, carries out display control of the article image as a result of obtaining the positional information of the article from the information identifying the article.

### BACKGROUND OF THE INVENTION

1. Field of the Invention

The present invention relates to an image reading system for storing a document image obtained from scanning, which document image can be read from a terminal.

2. Description of the Related Art

FIG. 1 shows a configuration of an image reading system in the related art, in which a document is scanned and input to a scanner apparatus 10 such as a MFP (Multi-Function Printer) or such (Step S1), the document is then registered, with a delivery destination designated, in a document management server 20 (Step S2). As a result, the document is delivered to a terminal 30 such as a PC (Personal Computer) or such from the document management server 20 (Step S3). Further, as a result of the terminal 30 accessing the document management server 20 (Step S4), search and reading of a document from the terminal is made possible (for example, see Japanese Laid-Open Patent Application 2005-208934).

In the image reading system in the related art, as mentioned above, the image is directly delivered to the terminal 30, i.e., the delivery destination. As a result, when an image which a user does not necessarily wish to read may also be delivered, a HDD (Hard Disk Drive) or such of the terminal 30 may be thus used uselessly, and also, the network load may increase.

Further, when a document image corresponds to such a document having a large page size as a newspaper or a document having many pages such as a book, which includes many articles, it may be difficult to know where a target article is actually placed there. This problem may become more remarkable especially for a mobile terminal having a small display area.

### SUMMARY OF THE INVENTION

The present invention has been devised in consideration of the above-mentioned problems, and, an object of the present invention is to provide an image reading system in which, it is possible to minimize a required display area or a network load, and, also, it is possible to make it easier to know where a target article is actually placed.

In order to solve the problems, according to the present invention, a part obtaining image data created from reading a document by means of a scanning function; a part creating positional information indicating an area of a particular article included in the image data; a part creating meta data including bibliographic information of the article, information identifying the article and the positional information; a part storing the thus-created meta data; a part delivering to a terminal at least the bibliographic information and the information identifying the article; a part delivering to the terminal the image data of the article in response to a request; and a part carrying out display control of the article image based on the positional information when the meta data has the positional information of the article, while, when the meta data does not have the positional information of the article, carrying out display control of the article image based on the positional information obtained from the information identifying the article, are provided.

Other objects and further features of the present invention will become more apparent from the following detailed description when read in conjunction with the accompanying drawings:

### BRIEF DESCRIPTION OF DRAWINGS

FIG. 1 shows a configuration of an image reading system in the related art;

FIG. 2 shows a configuration of an image reading system in one embodiment of the present invention;

FIG. 3 shows a general outline of processing carried out by the image reading apparatus in the embodiment of the present invention;

FIG. 4 shows a configuration of a document management server included in the image reading system in the embodiment of the present invention;

FIG. 5 shows a terminal also included in the image reading system in the embodiment of the present invention;

FIGS. 6 through 9 show examples of arrangements of functional parts;

FIG. 10 shows a sequence diagram for an example of image registration processing;

FIG. 11 shows a sequence diagram for an example of processing of positional information creation carried out by an editing terminal;

FIG. 12 shows a flow chart showing an example of positional information creation processing;

FIG. 13 shows one example of decomposing into document elements;

FIG. 14 shows an example in which a document image having a complicated layout is decomposed into document elements;

FIG. 15 shows an example in which document elements are grouped in article units;

FIG. 16 shows an example in which, by means of user's operation made from an operation panel of an MFP, document elements are grouped;

FIG. 17 shows an example of a user's operation for grouping document elements from a PC;

FIG. 18 shows an example in which document elements are selected from user's operation of toggling between designation and cancellation;

FIG. 19 shows an example of a state in which selection of document elements is completed;

FIGS. 20 and 21 show examples of document element selection by means of designation with setting a rectangle;

FIG. 22 shows an example of document element selection by means of designation with setting a line segment;

FIG. 23 shows an example of the entirety of a document image in which document element selection is finished;

FIG. 24 shows an example of a method for designating an article area;

FIG. 25 shows an example of expression of designating article range;

FIG. 26 shows an example of an RSS feed to be delivered;

FIG. 27 shows an example of description of control information;

FIG. 28 shows an example of processing for when the control information includes only an article number;

FIG. 29 shows an example of a multi-page image;

FIG. 30 shows an example of chapter/section/item configuration of a book;

FIGS. 31 through 33 show examples of RSS feeds to be delivered;

FIG. 34 shows an example of processing for when designation of page/coordinate is not carried out;

FIG. 35 shows a sequence diagram of an example of processing to obtain meta data;

FIG. 36 shows a sequence diagram of an example of processing for displaying an article;

FIG. 37 shows an example of reading page for when one article is included in one image and no positional control is used;

FIG. 38 shows an example of a reading page for when a plurality of articles are included in one image and positional control is carried out;

FIG. 39 shows an example of page scrolling control; and

FIG. 40 shows an example of display control for those other than a target article.

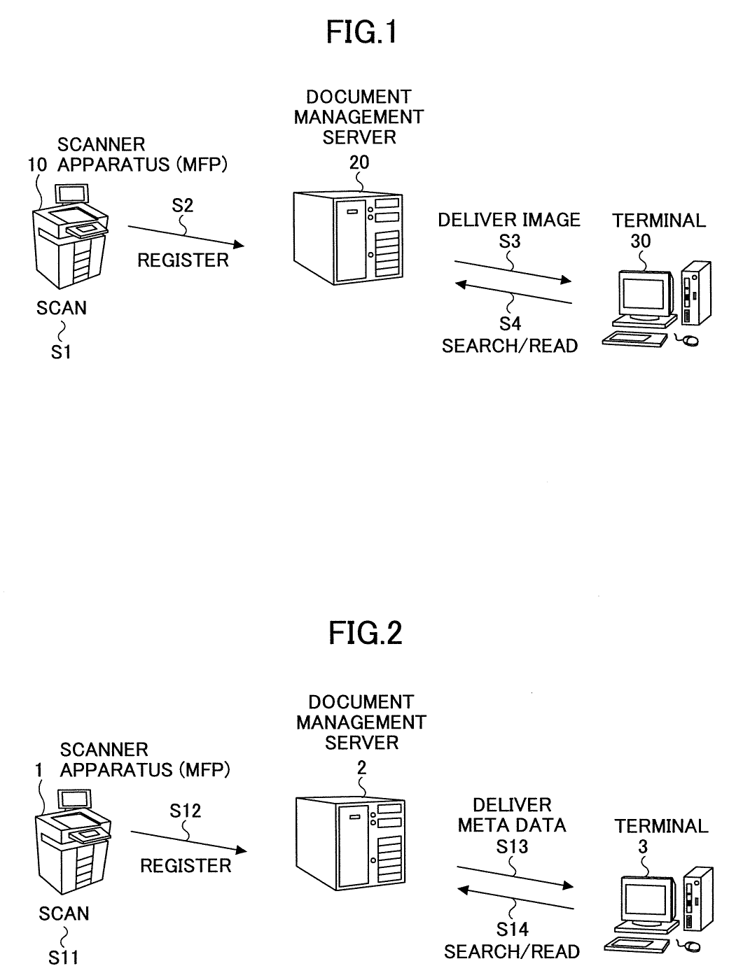

### DETAILED DESCRIPTION OF THE PREFERRED EMBODIMENTS

According to a preferable embodiment of the present invention, an image reading system has a part obtaining image data created from reading a document by means of a scanning function; a part creating positional information indicating an area of a particular article included in image data; a part creating meta data including bibliographic information of the article, information identifying the article and the positional information; a part storing the thus-created meta data; a part delivering to a terminal at least the bibliographic information and the information identifying the article; a part delivering to the terminal the image data of the article in response to a request; and a part carrying out display control of the article image based on the positional information when the meta data has the positional information of the article, while, when the meta data does not have the positional information of the article, carrying out display control of the article image based on the positional information obtained from the information identifying the article.

In the image reading system, the image data to be delivered to the terminal may have the entirety of the image data including the article actually required.

In the image reading system, the image data to be delivered to the terminal may have an area of the article extracted from the image data based on the positional information of the article.

In the image reading system, the creation of the positional information may be carried out based on a position of an area including document elements, obtained from grouping of the document elements which are included in one article, from among those obtained from decomposing an image included in the image data.

In the image reading system, the grouping may be of selecting of the document elements.

In the image reading system, the selecting of the document elements may be carried out by means of operation for toggling between designation and cancellation.

In the image reading system, the selecting of the document elements may be carried out by means of setting a rectangle, and selecting the document elements included therein.

In the image reading system, the selecting of the document elements may be carried out by means of setting a line segment, and selecting the document elements crossed by the line segment.

In the image reading system, the grouping may be carried out from designating of an article area made by a user.

In the image reading system, the display control may include detecting a position corresponding to a top of an article image.

In the image reading system, the display control may include limiting a range for which strolling is allowed, in a target article area.

In the image reading system, the display control may include masking those other than a target article.

A document management server according to a preferable embodiment of the present invention has a part obtaining image data created from reading a document by means of a scanning function; a part creating positional information indicating an area of a particular article included in the image data; a part creating meta data including bibliographic information of the article, information identifying the article and the positional information; a part storing the thus-created meta data; a part delivering to a terminal at least the bibliographic information and the information identifying the article; and a part delivering to the terminal the image data of the article in response to a request.

In the document management server, the image data to be delivered to the terminal may be the entirety of the image data including the article required.

In the document management server, the image data to be delivered to the terminal may have an area of an article extracted from the image data based on the positional information of the article requested.

In the document management server, the creation of the positional information may be carried out based on a position of an area including document elements, obtained from grouping the document elements which are included in one article, from among those obtained from decomposing an image included in the image data.

In the document management server, the grouping may be of selecting of the document elements.

In the document management server, the grouping may be carried out from designating of an article area made by a user.

An image reading control method according to a preferable embodiment of the present invention has the step of obtaining image data created from reading a document by means of a scanning function; the step of creating positional information indicating an area of a particular article included in the image data; the step of creating meta data including bibliographic information of the article, information identifying the article and the positional information; the step of storing the thus-created meta data; the step of delivering to a terminal at least the bibliographic information and the information identifying the article; the step of delivering to the terminal the image data of the article in response to a request; and the step of carrying out display control of the article image based on the positional information when the meta data has the positional information of the article, while, when the meta data does not have the positional information of the article, carrying out display control of the article image based on the positional information obtained from the information identifying the article.

An image reading control program according to a preferable embodiment of the present invention has instructions for causing a computer to act as means for obtaining image data created from reading a document by means of a scanning function; means for creating positional information indicating an area of a particular article included in the image data; means for creating meta data including bibliographic information of the article, information identifying the article and the positional information; means for storing the thus-created meta data; means for delivering to a terminal at least the bibliographic information and the information identifying the article; means for delivering to the terminal the image data of the article in response to a request; and means for carrying out display control of the article image based on the positional information when the meta data has the positional information of the article, while, when the meta data does not have the positional information of the article, carrying out display control of the article image based on the positional information obtained from the information identifying the article.

According to the embodiments of the present invention described above, it is possible to minimize a required storage area and a required network load, and also, it is possible to make it easier to know a place of a target article.

Below, the preferable embodiments of the present invention will be described in more detail.

<System Configuration>

FIG. 2 shows a configuration example of an image reading system in one embodiment of the present invention.

In FIG. 2, the image reading system includes a scanner apparatus 1 such as an MFP including an image obtaining part, a document management server 2 carrying out storage of a document image, creation and/or delivery of meta data, or such, and a terminal 3 such as a PC which a user operates. Further, in a general processing flow, the scanner apparatus 1 scans a document (Step S11), and the document management server 2 registers the same (Step S12). Thereby, from the document management server 2, to the terminal 3, meta data including an article summary (i.e., bibliographic information and information specifying an article) and control information (positional information and so forth) is delivered, by means of an RSS (Rich Site Summary) feed, an Atom delivery or such(Step S13). In the terminal 3, from the meta data, the article is determined, which is then accessed by the document management server 2, thus, a relevant document image is obtained, and thus, search and/or reading of the article is achievable (Step S14).

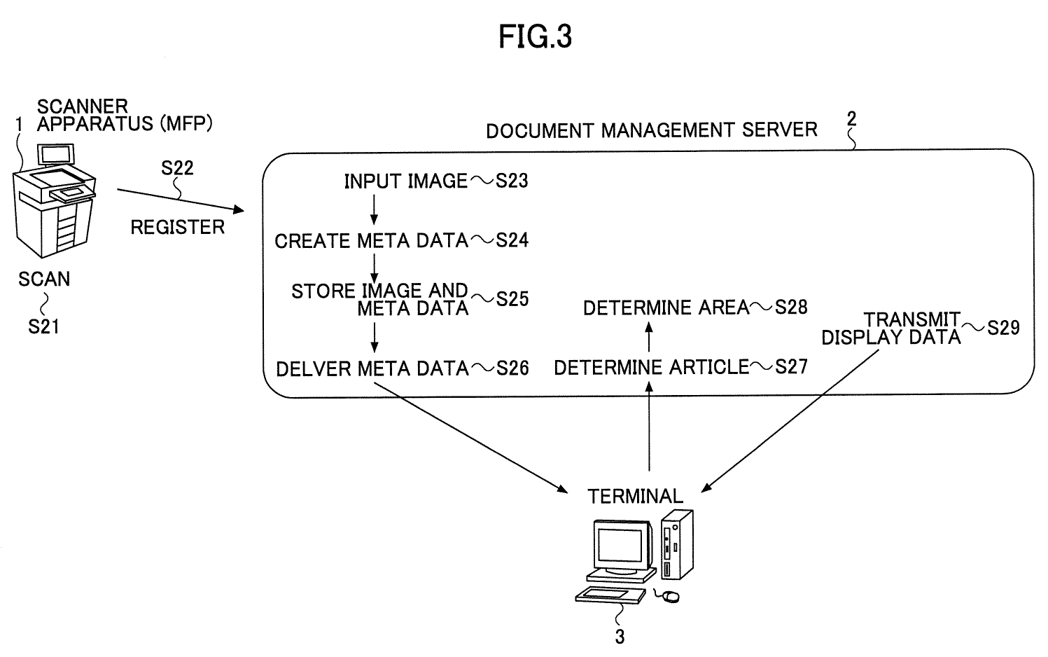

FIG. 3 shows a general outline of processing carried out by the image reading system. The scanner apparatus 1 scans a document (Step S21), the document is registered in the document management server 2 (Step S22), and thus, the document management server 2 carries out, in sequence, processing of the input image (Step S23), processing of meta data creation (Step S24), processing of storage of the image and the meta data (Step S25), and processing of delivery of the meta data (Step S26). In the processing of the meta data creation (Step S24), by means of manual processing or automatic processing with an OCR (Optical Character Reader) or such, positional information, bibliographic information (a title, a search keyword, an abstract/summary and so forth) are extracted or created, and thus, the meta data is created.

The terminal 3 which thus receives the meta data then may access the document management server 2. Thereby, a target article is determined (Step S27), a corresponding area is determined (Step S28), and corresponding display data is transmitted (Step S29), in sequence.

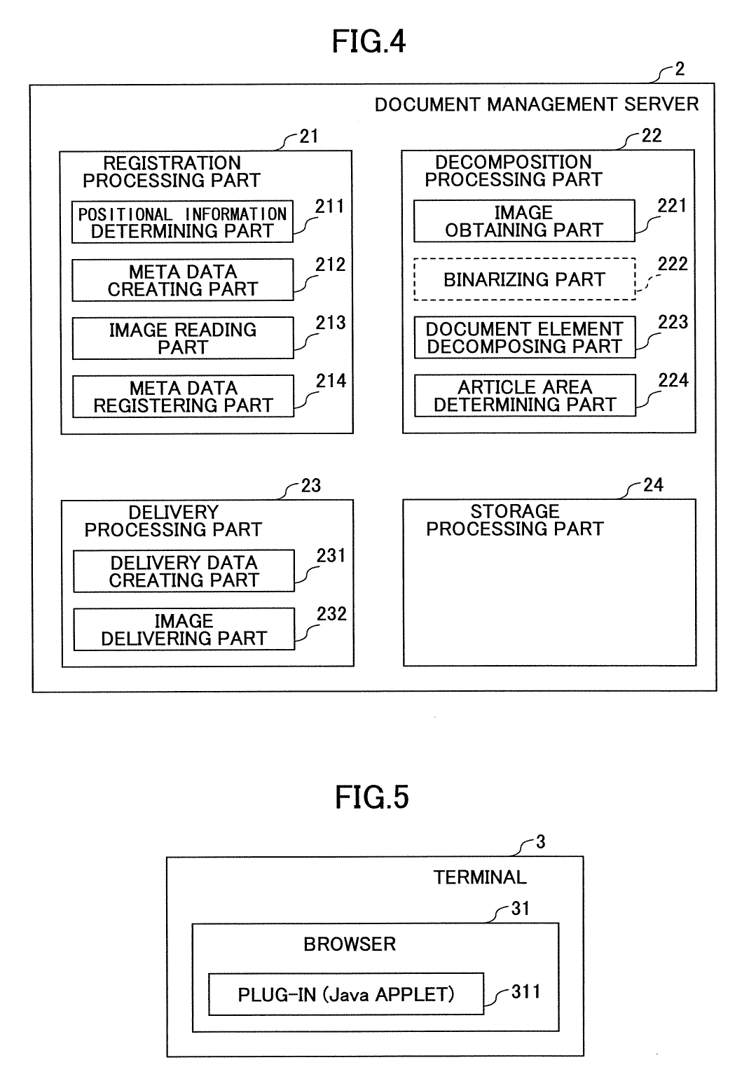

FIG. 4 shows a configuration example of the document management server 2. The document management server 2 includes a registration processing part 21 for registering a document image and meta data, a decomposition processing part 22 for carrying out decomposition processing for determining positional information of an article from a document image, a delivery processing part 23 for carrying out delivery of the meta data and the document image, and a storage processing part 24 for carrying out storage of the document image and the meta data. It is noted that, as to functions carrying out network communication and overall control, indication/description of corresponding parts are omitted for the purpose of simplicity of description.

The registration processing part 21 includes a positional information determining part 211 for determining, by means of automatic processing or manual operation by a user who registers a document image, positional information of an article area, a meta data creating part 212 for creating meta data by means of automatic processing or manual input operation by the user, an image registering part 213 for registering a document image in the storage processing part 24, and a meta data registering part 214 for registering the meta data in the storage processing part 24.

The decomposition processing part 22 includes an image obtaining part 221 for obtaining a document image on which decomposition processing is carried out, a binarizing part 22 (which is not necessary when binarization is not carried out, and thus, multi-level image is processed as it is) for binarizing the document image, a document element decomposing part 223 for decomposition the document into document elements such as characters, lines, columns or such, and an article area determining part 224 determining an article area by grouping the document elements in article units by means of automatic processing or manual operation by a user.

The delivery processing part 23 includes a delivery data creating part 231 creating delivery data such as an RSS feed or such, from the meta data, and an image delivery part 232 delivering a document image (which may include, as is necessary, control information).

FIG. 5 shows an example of a configuration of the terminal 3. In the terminal 3, mainly in a reading terminal, a browser 31 is provided such that delivery data including the meta data delivered by means of an RSS feed or such, is received, a target article may be selected from a list of articles, and thus, a corresponding image part is designated, and a plug-in 311 such as Java applet or such, is provided whereby display control such as detecting a position corresponding to a top of the target article or such is carried out.

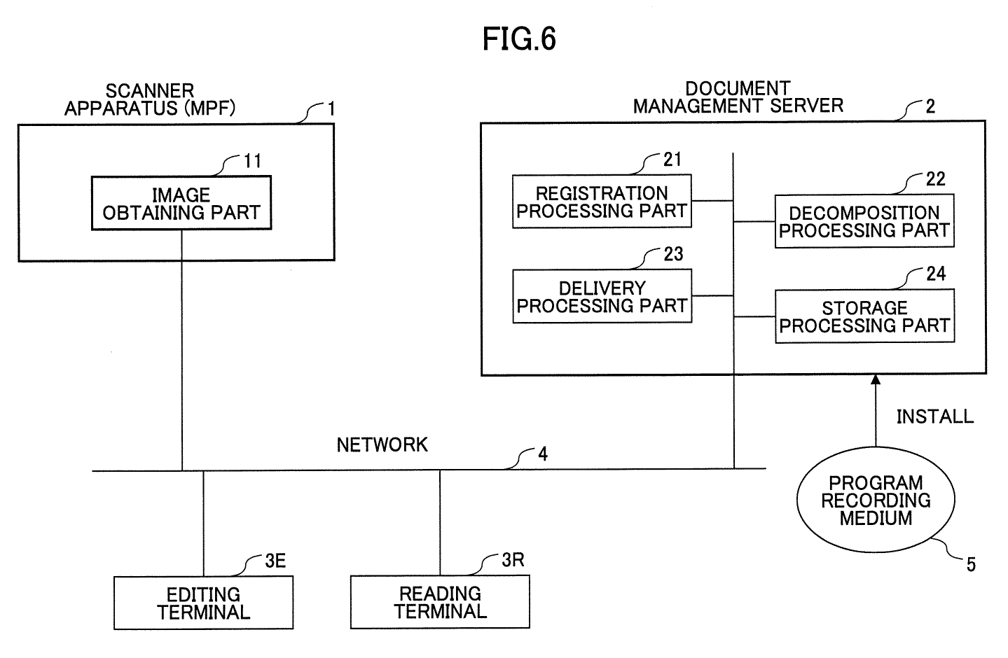

FIGS. 6 through 9 show examples of arrangements of functional parts. FIG. 6 corresponds to FIG. 2. In the arrangement of FIG. 6, the scanner apparatus 1 including the image obtaining part 11, the document management server 2, an editing server 3E, and a reading terminal 3R are arranged in a communication network 4. It is noted that the respective functional parts of the document management server 2 are actually realized as a result of a corresponding computer program being installed from a program recording medium 5.

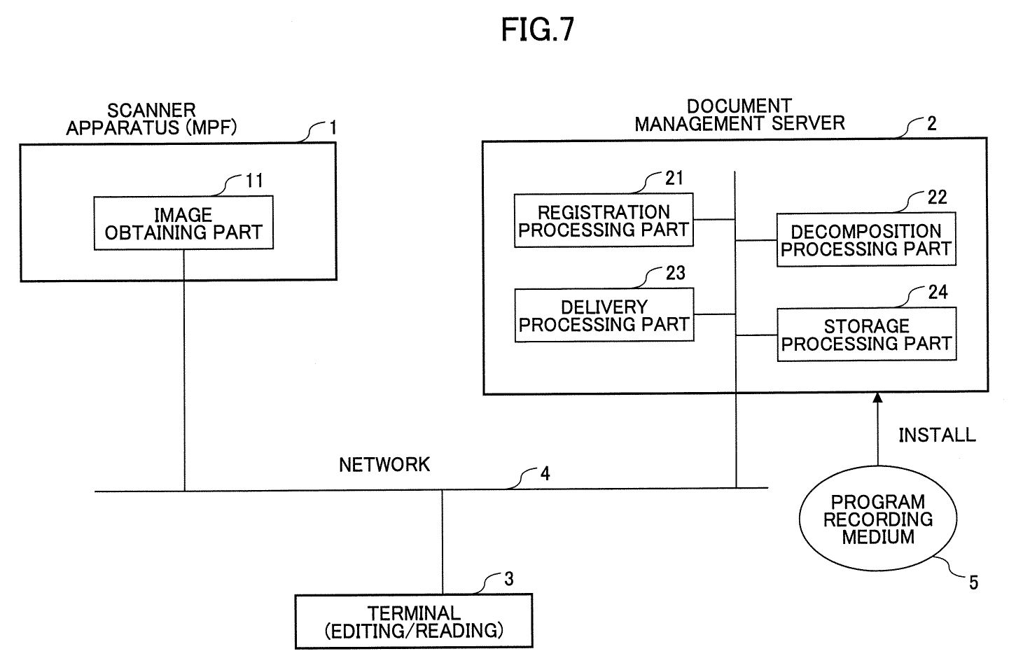

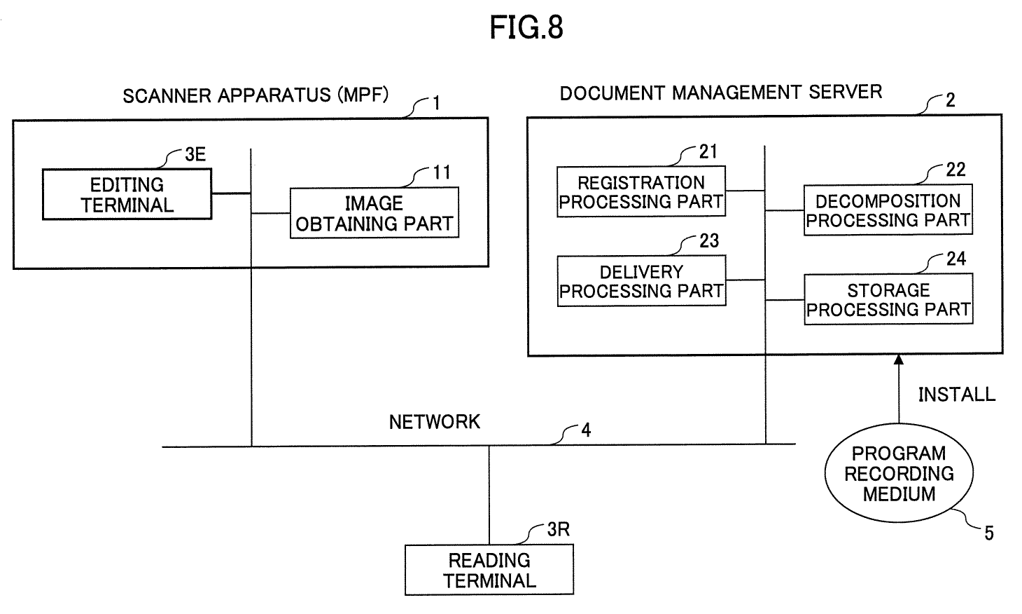

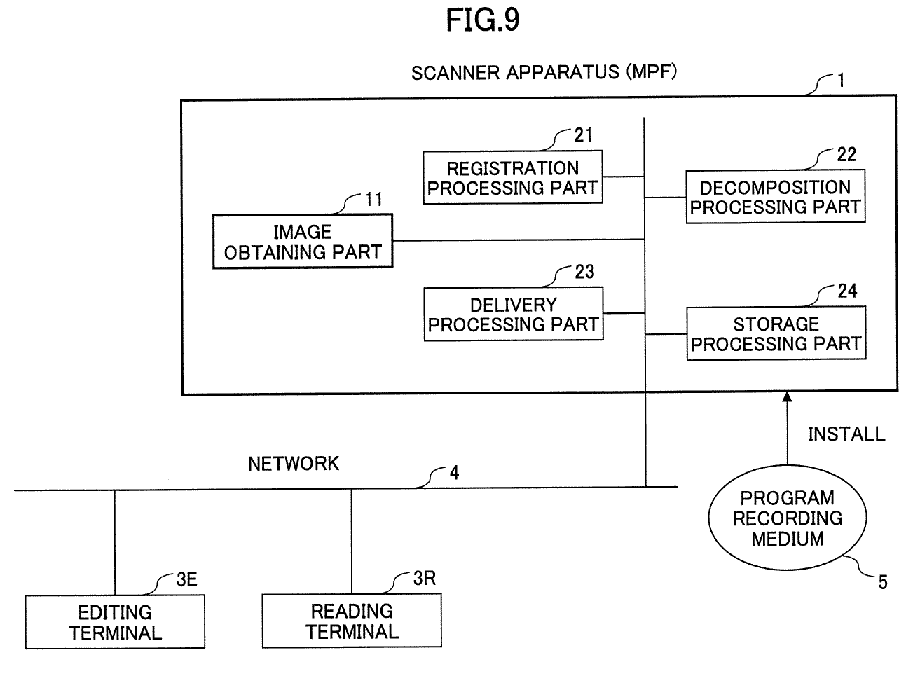

FIG. 7 shows a variant example, in which the terminal 3 acts both as the editing terminal 3E and as the reading terminal 3R. FIG. 8 shows an example in which, in the scanner apparatus 1, the function of the editing terminal 3E is included. FIG. 9 shows an example in which, in the scanner apparatus 1, the function of the document management server 2 is included.

<Processing of Scanning Image for Registration>

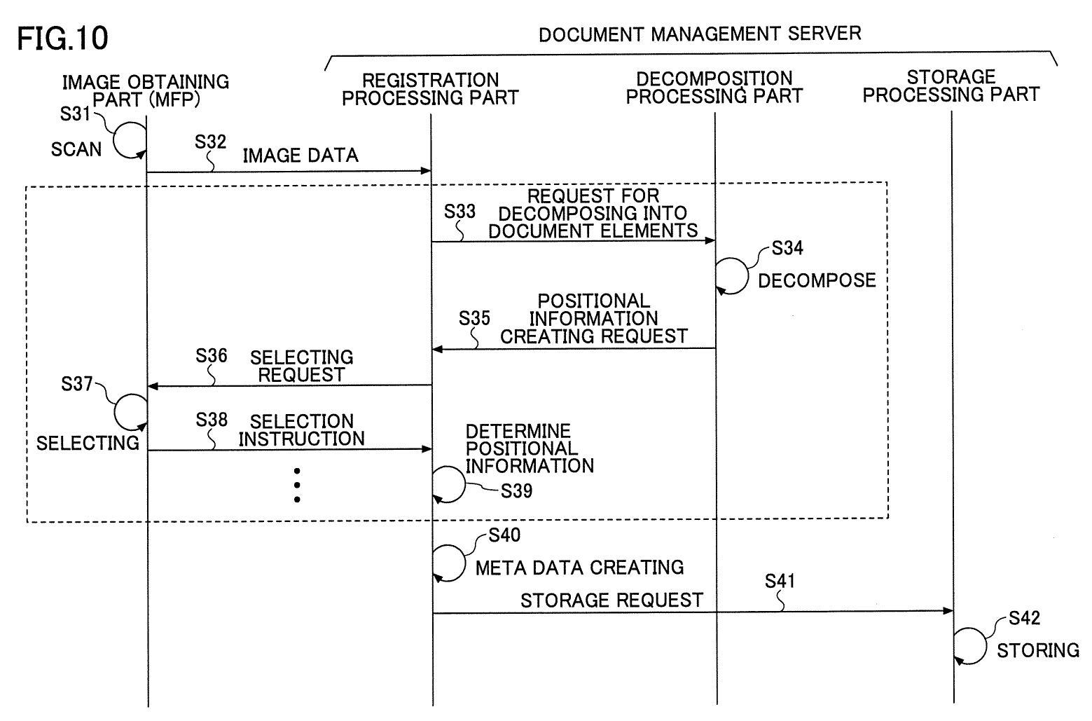

FIG. 10 shows a sequence diagram showing a processing example of image registration.

In FIG. 10, the scanner apparatus 1 scans a document (Step S31), the thus-obtained image data is transmitted to the registration processing part 21 of the document management server 2 (Step S32), and the registration processing part 21 requests the decomposition processing part 22 to decompose the document data into document elements (Step S33).

The decomposition processing part 22 carries out decomposition of the given document image into document elements (Step S34), and requests the registration processing part 21 to create positional information (Step S35).

The registration processing part 21 requests the scanner apparatus 1 to select a document element included in a target article from among the thus-decomposed document elements (Step S36). From the scanner apparatus 1, a user selects the document element on an operation panel of the scanner apparatus 1 (Step S37), and the scanner apparatus 1 transmits the thus-obtained selection instruction to the registration processing part 21 (Step S38). This operation of selecting the document element (Step S37, S38) may be repeated as is necessary.

The registration processing part 21 determines positional information of an article based on the selection instruction (Step S39), creates meta data including the positional information (Step S40), requests the storage processing part 24 to store it (Step S41), and the storage processing part 24 stores the image data and the meta data (Step S42).

It is noted that, in FIG. 10, the processing enclosed by a broken line, i.e., Steps S33 through S39, may not be carried out, and, thus, only the image data and the meta data (not including the positional information) may be stored. In this case, creation of the article's positional information is carried out in processing of positional information creation carried out by the editing terminal described next.

<Processing of Positional Information Creation from Editing Terminal>

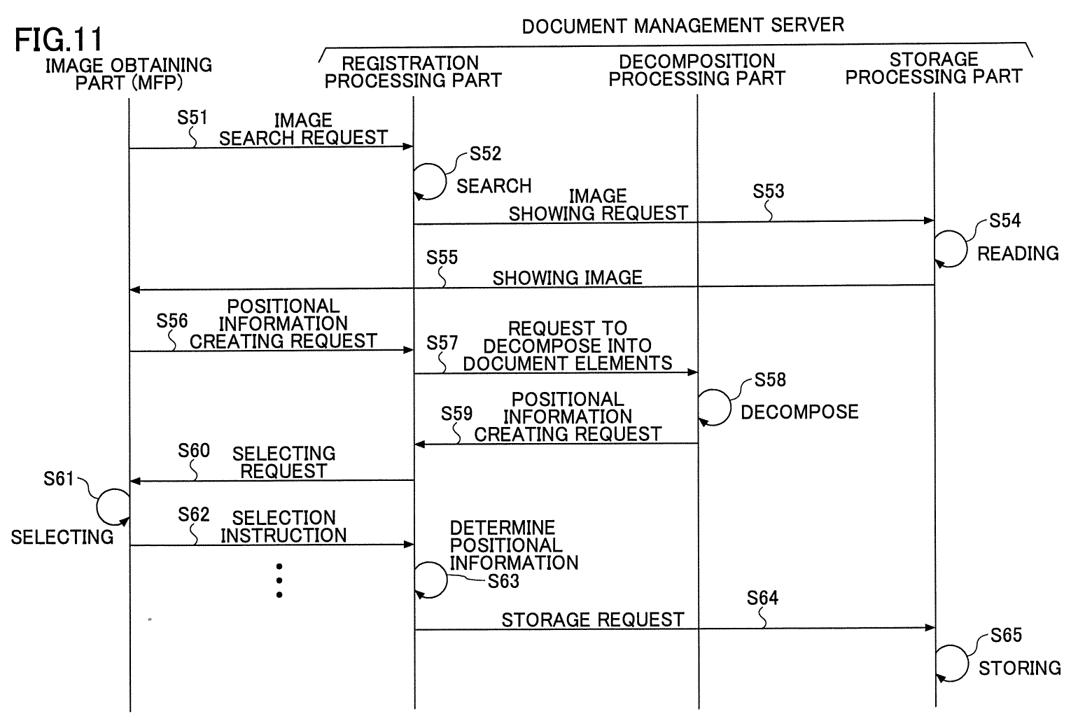

FIG. 11 shows a sequence diagram showing an example of processing of positional information creation made from the editing terminal 3E.

In FIG. 11, when the editing terminal 3E requests the registration processing part 21 of the document management server 2 to create positional information (Step S51), the registration processing part 21 carries out search (Step S52), and requests the storage processing part 24 to show a document image (Step S53). The storage processing part 24 receiving this request shows document image data, obtained from the search, to the editing terminal 3E (Step S55) as a result of reading the document image data (Step S54).

Next, when the editing terminal 3E requests the registration processing part 21 to create positional information (Step S56), the registration processing part 21 requests the decomposition processing part 22 to decompose the given document image into document elements (Step S57).

The decomposition processing part 22 carries out decomposition of the document image into document elements (Step S58, and requests the registration processing part 21 to create positional information (Step S59).

The registration processing part 21 requests the editing terminal 3E to select a document element included in a target article from among the thus-decomposed document elements (Step S60), and the editing terminal 3E selects the document element (Step S61), and transmits a corresponding selection instruction to the registration processing part 21 (Step S62). This processing for selection (Steps S61, S62) may be repeated as is necessary.

The registration processing part 21 determines positional information of an article from the selection instruction (Step S63), requests the storage processing part 24 to store the same (Step S64), and the storage processing part 24 stores the positional information as a part of the meta data (Step S65).

<Details of Positional Information Creation>

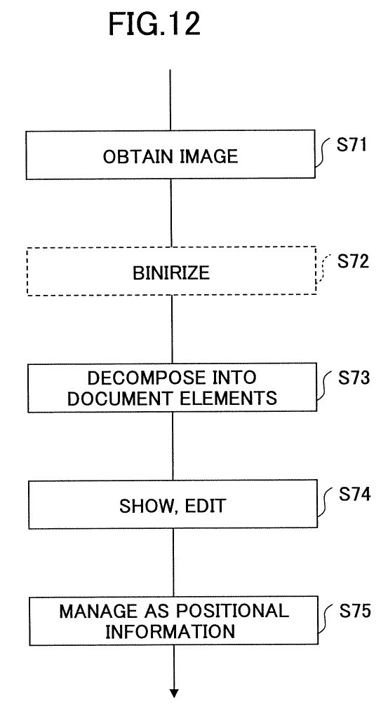

FIG. 12 shows a flow chart of an example of processing of the positional information creation, and, shows the processing of FIG. 10 (Steps S33 through S39) and the processing of FIG. 11 (Steps S57 through S63), in common.

In FIG. 12, first, target document image data for positional information creation is obtained (Step S71). The image data may be one of document image data immediately after being scanned or document image data already stored.

Next, as previous processing for making it easier to decompose the document t image into document elements, the given document image data is binarized (Step S72). It is noted that, this processing is not necessary when a multi-level image is processed as it is without being thus binarized.

Next, with attention to characters, figures or such included in the document image data, decomposition of the document image data to document elements is carried out (Step S73).

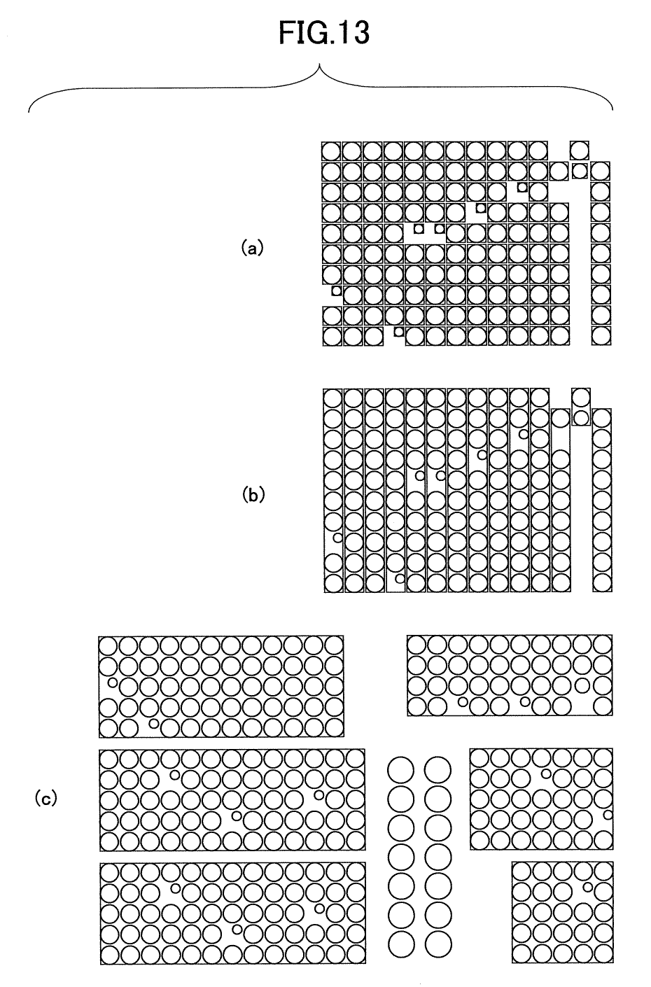

FIG. 13 shows examples of decomposition of the given document image data into document elements. FIG. 13, (a) shows an example, in which decomposition is made in character units, FIG. 13, (b) shows an example in which decomposition is made in line units (in a case of a vertical writing document, and FIG. 13, (c) shows an example in which decomposition is made in column units. With the use of the decomposition result in characters units shown in FIG. 13, (a), it is possible to carry out combination for the decomposition result in line units shown in FIG. 13, (b). Similarly, it is possible to carry out combination for the decomposition result in column units shown in FIG. 13, (c) with the use of the decomposition result in line units shown in FIG. 13, (b). It is also possible to carry out decomposition directly into each of the respective states of FIG. 13, (a), (b) and (c) from the given document image data. It is noted that, it is possible to determine whether or not given character lines/columns correspond to a vertical writing document or a horizontal writing document, with the use of the decomposition result in line units.

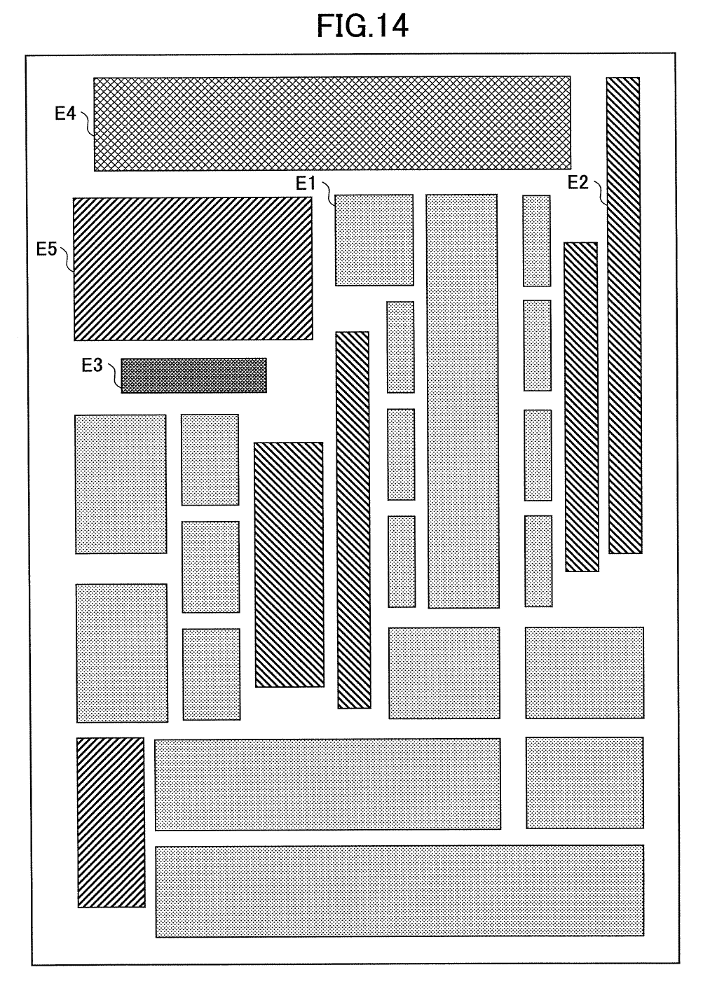

FIG. 14 shows an example in which a document image having a complicated layout. A document element El and document elements having the same hatching show vertically written sentence parts, a document element E2 and document elements having the same hatching show vertically written title and subheading parts, a document element E3 shows a horizontal sentence part, a document element E4 shows horizontal writing title and subheading, and a document element E5 and a document element having the same hatching show photograph and/or drawing parts.

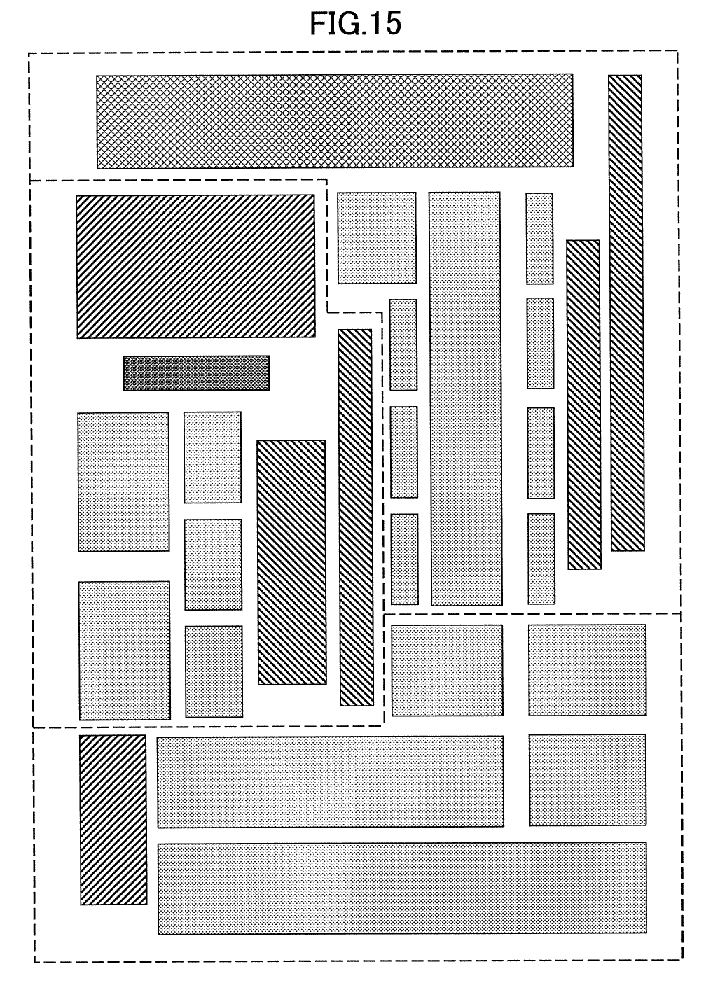

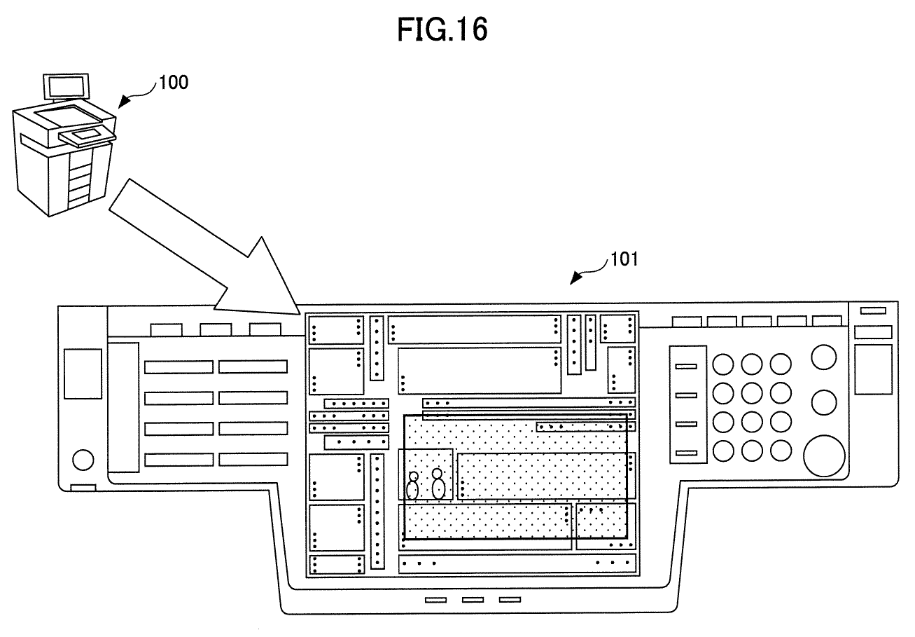

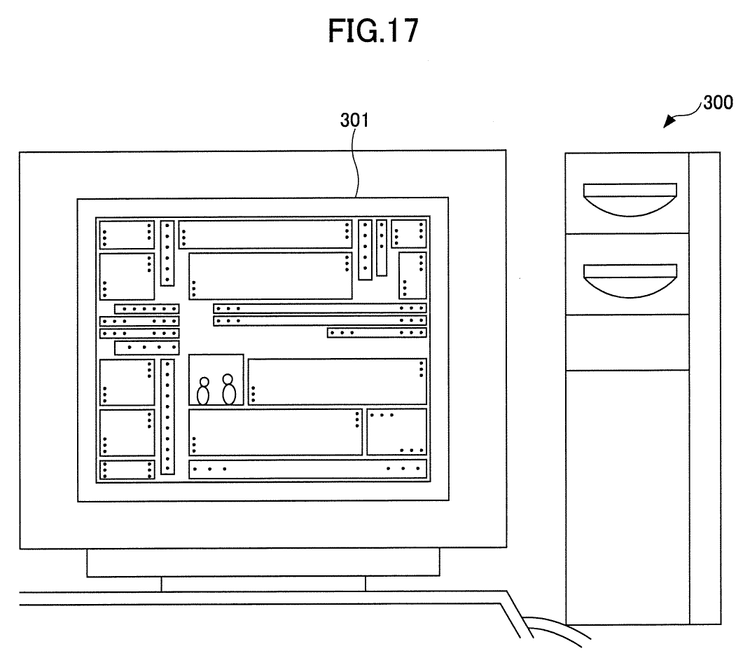

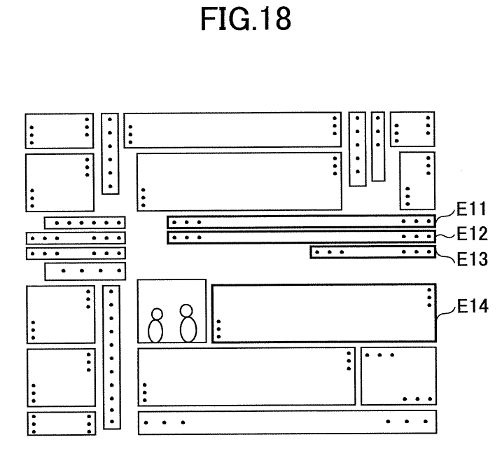

Next, returning to FIG. 12, the thus-decomposed document elements are shown to a user, and thus, the user is made to edit, by grouping the shown document elements included in a target article (Step S74). FIG. 15 shows an example in which the document elements are grouped in article units. In FIG. 15, the document elements enclosed by a broken line correspond to one article. FIG. 16 shows an example of operation of grouping of the document elements from an operation panel 101 of an MFP 100 (as the above-mentioned scanner apparatus 1). FIG. 16 shows a state in which document elements are displayed on the operation panel 101 provided with a large-sized touch-panel-type display device. FIG. 17 shows an example of operation of grouping of the document element by means of a PC 300, and shows a state in which, on a monitoring screen 301, the document elements are displayed. It is noted that, when the grouping processing is carried out completely automatically, the above-mentioned processing is not necessary.

As specific examples of the selection operation for the grouping, the following operation may be made by means of touching a panel or clicking a mouse by an operator:

(1) document element selection by means of toggling operation between assignment and cancellation;

(2) document element selection by means of setting a rectangle; and

(3) document element selection by means of setting a line segment.

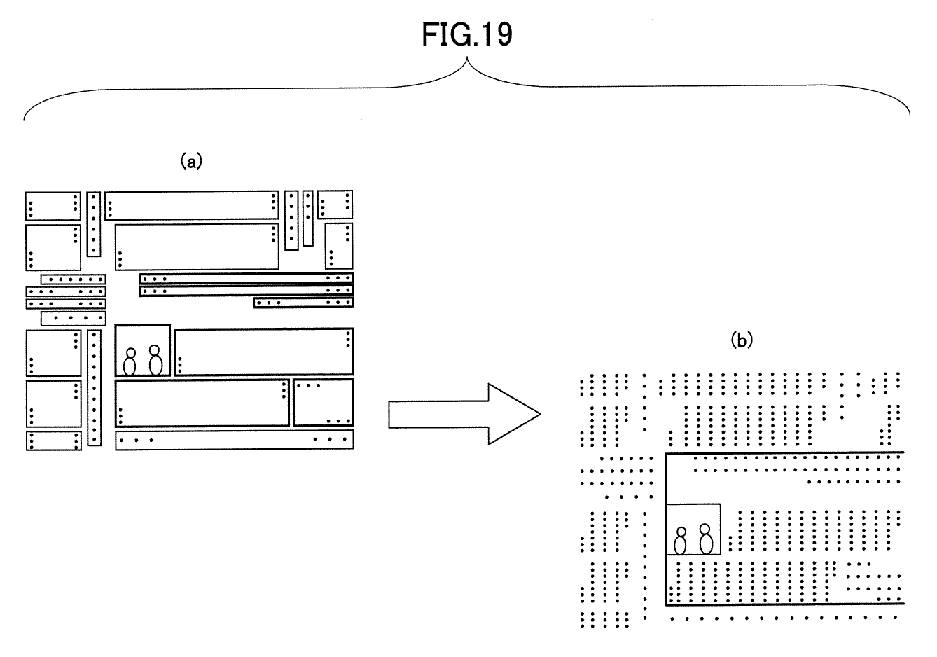

FIG. 18 shows an example of the above-mentioned document element selection by means of the toggling operation between designation and cancellation. In FIG. 18, document elements E11 through E14 indicated with bold lines show respective designated states. By clicking respective ones of the thus-designated document elements again, it is possible to cancel the designation states. FIG. 19, (a) shows a state in which selection of document elements, indicated with bold lines, has been completed. In FIG. 19, (a), the entirety of the designated document elements are recognized as one article as enclosed by a bold line, as shown in FIG. 19, (b).

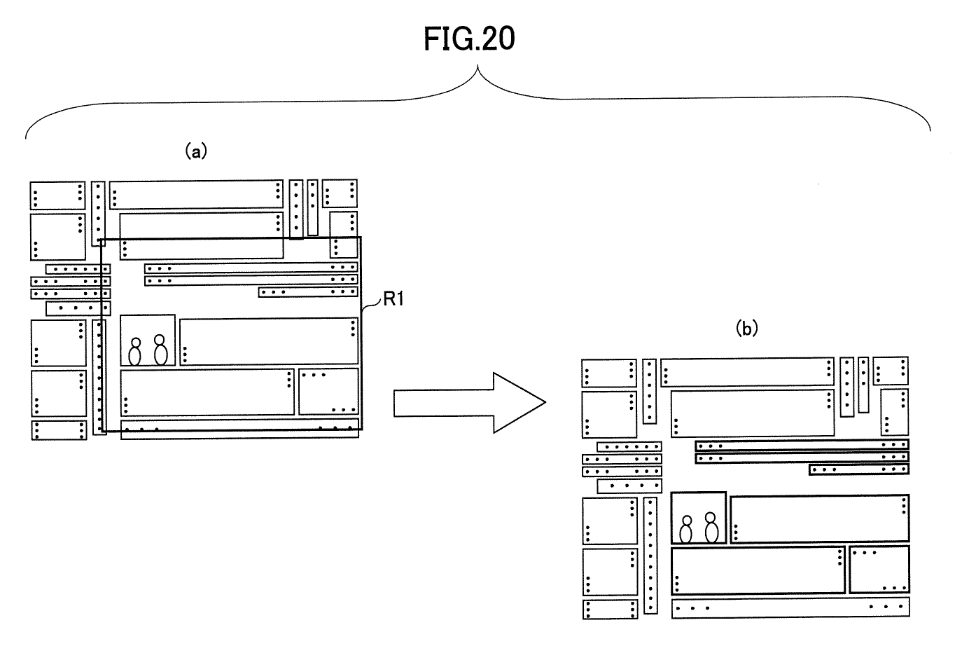

FIG. 20 shows an example of the above-mentioned document element selection by means of setting a rectangle. As shown in FIG. 20, (a), by means of setting a rectangle R1, it is possible to designate all the document elements included in the rectangle R1 as indicated with bold lines in FIG. 20, (b). Thus, these document elements are recognized as one article. It is noted that, the setting of this rectangle R1 may be made, not so strictly but somewhat roughly. Then, after that, if any, some document elements to be designated are missed or some document elements are erroneously designated, designation/cancellation may be made, one by one, separately, by means of the above-mentioned toggling between assignment and cancellation for example.

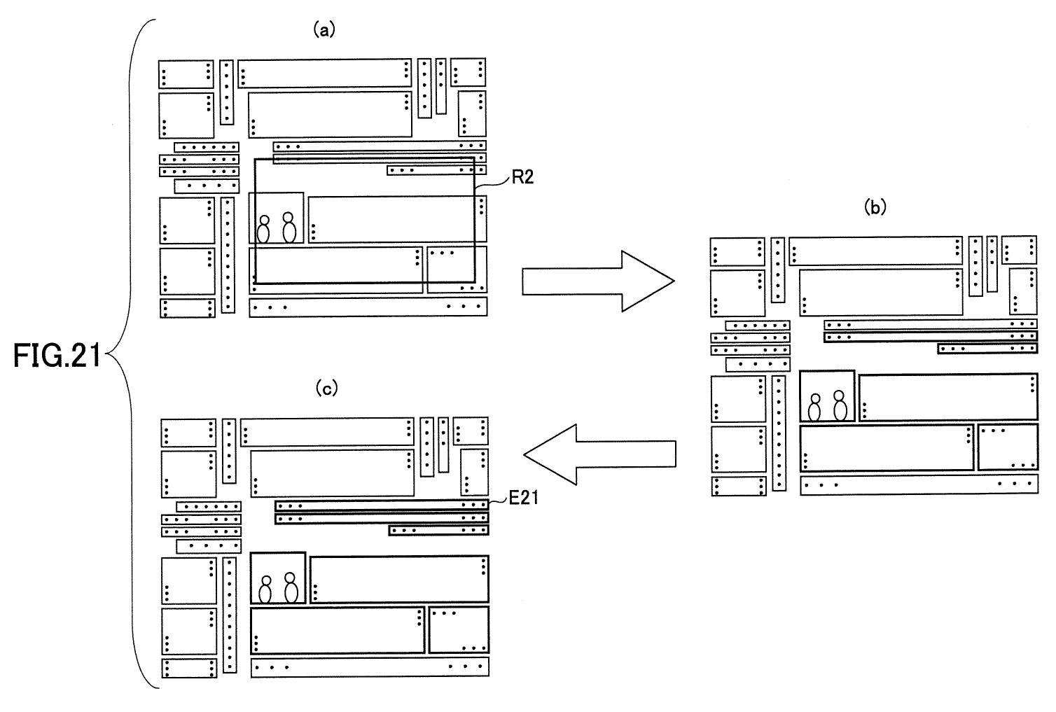

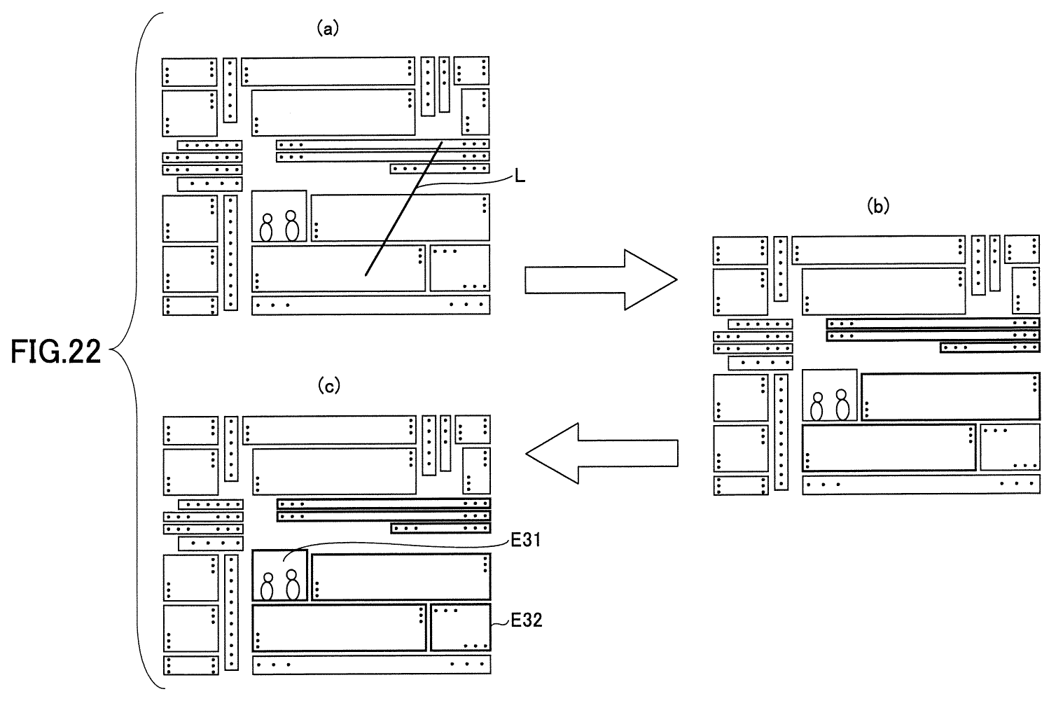

FIG. 21 shows another example of document element selection by means of setting a rectangle. As shown in FIG. 21, (a), by setting a rectangle R2, it is possible to designate all the document elements each of which is included in the rectangle R2 at least a part thereof, as indicated with bold lines in FIG. 21, (b) these document elements are then recognized as one article. It is noted that, the setting of this rectangle R2 may be made, not so strictly but somewhat roughly. Then, after, if any, some document elements to be designated are thus missed or some document elements are erroneously designated, designation/cancellation may be made one by one separately, by means of the above-mentioned toggling between assignment and cancellation for example. FIG. 21, (c) shows a state in which, a document element E21 which is missed being designated is then designated separately after that.

FIG. 22 shows one example of document element selection by means of setting a line segment. As shown in FIG. 22, (a), by setting a line segment L, it is possible to designate all the document elements crossed by the line segment L, as shown in FIG. 22, (b) with bold lines. These document elements are then recognized as one article. It is noted that, the setting of this line segment L may be made not so strictly but somewhat roughly. Then, after, if any, some document elements to be designated are thus missed or some document elements are erroneously designated, designation/cancellation may be made separately, by means of the above-mentioned toggling between assignment and cancellation for example. FIG. 22, (c) shows a state in which document elements E31 and E32 which are missed being designated are then designated separately.

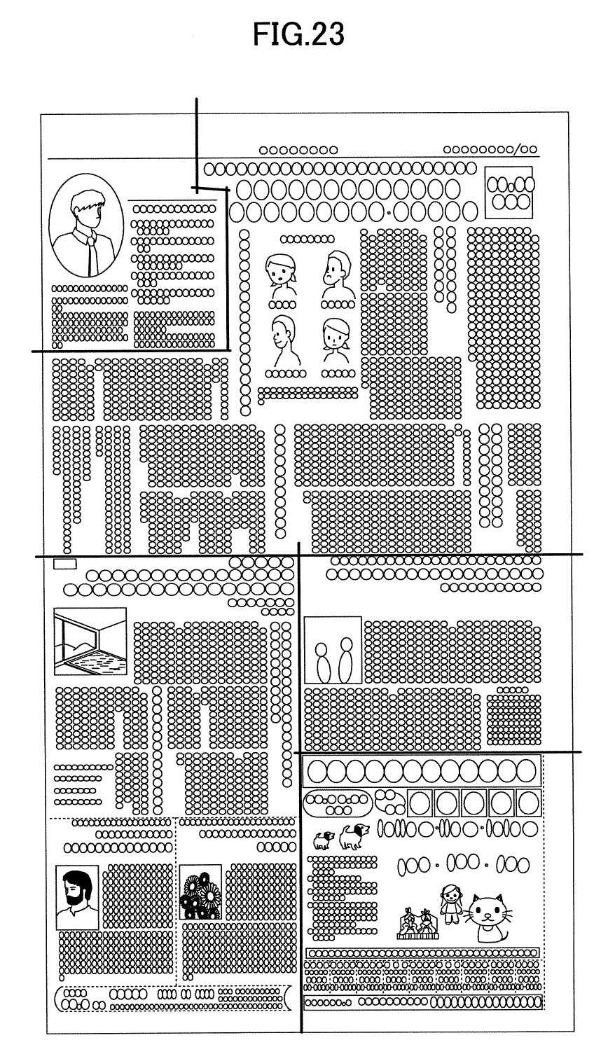

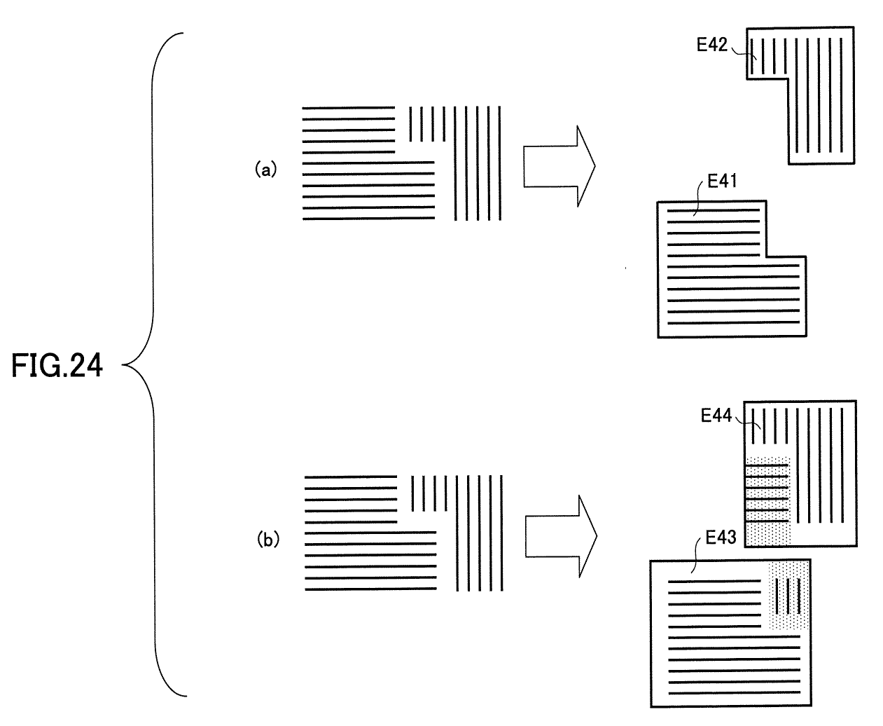

FIG. 23 shows an example of the entirety of a document image in which document element selection is completed. FIG. 23 shows a state in which dividing is made into articles as separated by bold lines. It is noted that, instead of the above-described methods, the articles may be divided, as a result of a user himself or herself directly carrying out designation/dividing corresponding to the bold lines.

FIG. 24 shows an example of a specific method for article selection. Originally, as shown in FIG. 24, (a), left articles should be divided into complicated document elements E41 and E42. However, for when management of such a way other than with setting a simple rectangle is difficult, simple rectangle document elements E43, E44 shown in FIG. 24, (b) are designated first, and after that, masking may be made for adjacent article parts, as hatched, when they are actually displayed, to achieve the same effect.

Next, returning to FIG. 12, an article area thus defined by means of the grouping is managed as positional information (Step S75).

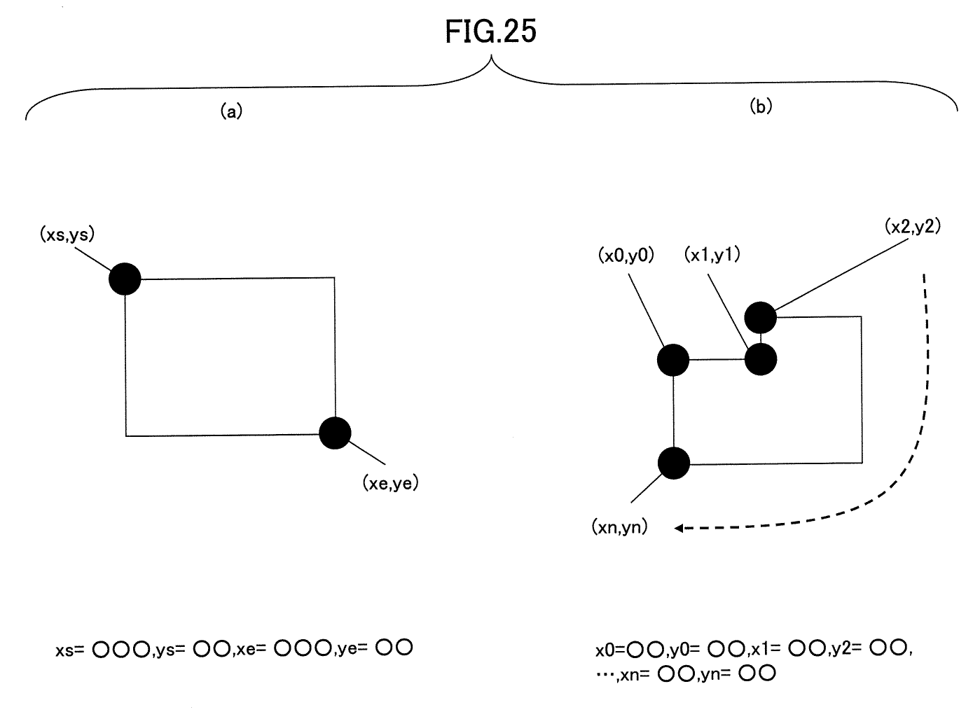

FIG. 25 shows an example of expression of such an article area. FIG. 25, (a) shows an example in which, by listing x-coordinates and y-coordinates of a top left corner and a bottom-right corner, the article area is expressed. FIG. 25, (b) shows an example in which, by listing x-coordinates and y-coordinates of a plurality of corners, the article area is expressed.

<Examples of Delivery Data created from Meta Data>

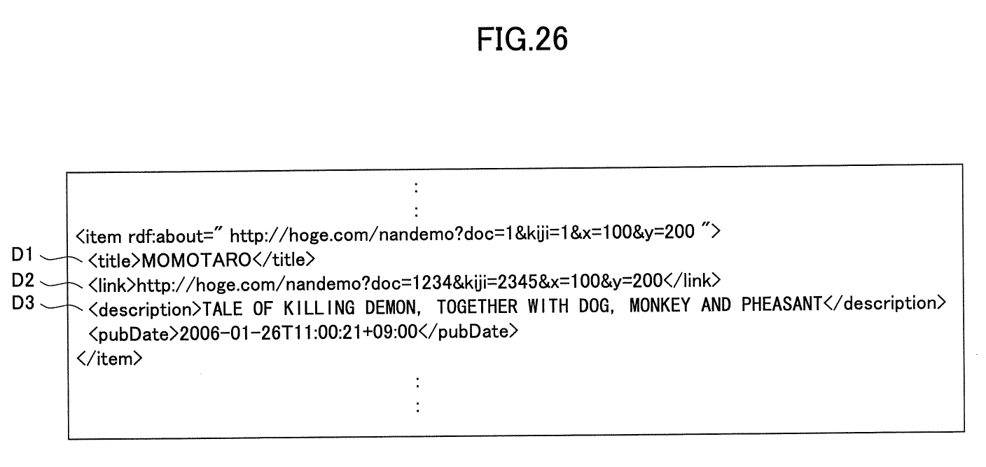

FIG. 26 shows an example of an RSS feed delivered. Below an item tag, a title description D1 which is indicated by a title tag, a description D2 of an access destination URL (Uniform Resource Locator), article number/positional information and so forth, which are indicated by a link tag, and a summary description D3 indicated by a description tag, are included.

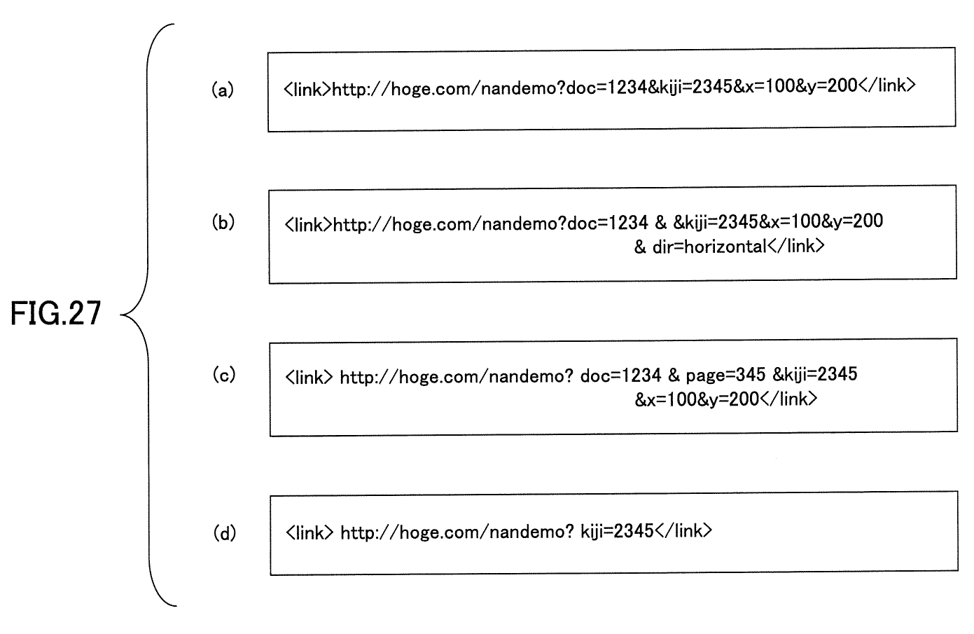

FIG. 27 shows an example of a description of control information included in the link tag. FIG. 27, (a) shows an example in which a document number doc, an article number kiji and coordinates x, y are included. FIG. 27, (b) shows an example in which a document number doc, an article number kiji, coordinates x, y, and a line direction dir (in which horizontal means horizontal writing; and vertical means vertical writing) are included. FIG. 27, (c) shows an example in which a document number doc, a page number page, an article number kiji, coordinates x, y are included. FIG. 27, (d) shows an example in which only an article number kiji is included.

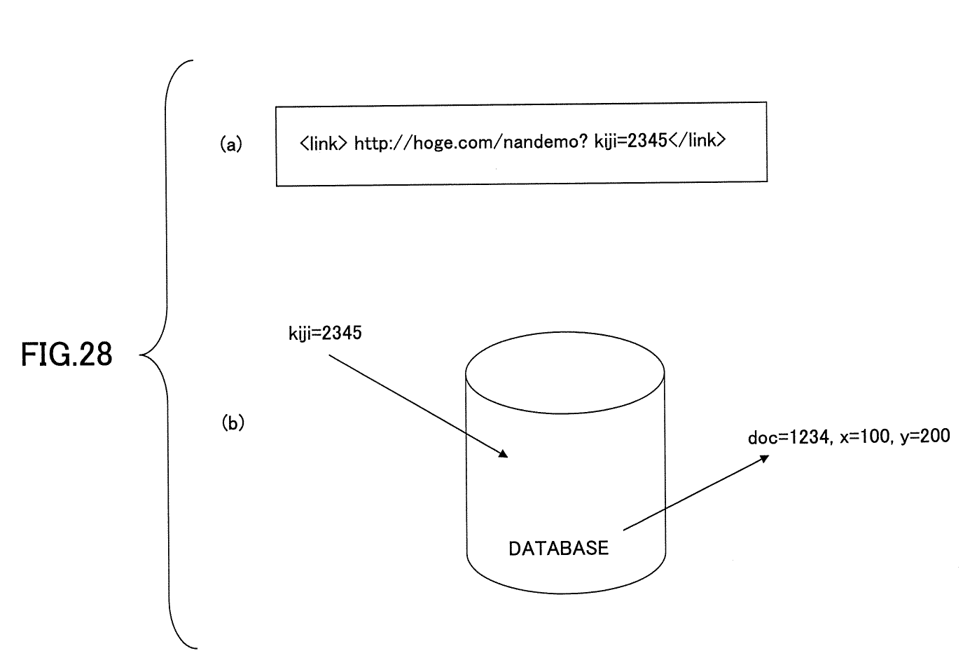

FIG. 28 shows an example of processing for a case where the control information includes only the article number kiji, as shown in FIG. 27, (d). As shown in FIG. 28, (a), when access to the document management server 2 is made based on the link tag, the document number doc and the coordinates x, y are determined from a database, based on the article number kiji, as shown in FIG. 28, (b).

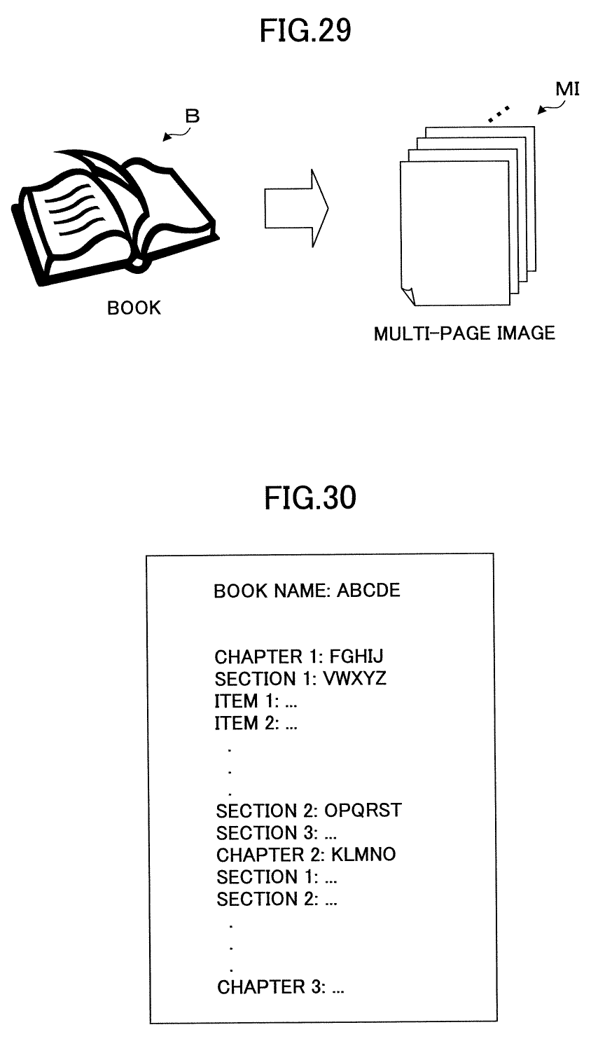

FIGS. 29 through 39 show examples of applications to multi-page images. A multi-page image is such that a plurality of pages are included in one image file. For example, a multi-page TIFF (Tagged Image File Format) exists. FIG. 29 shows an example of a multi-page image. As a result of scanning a book B, images thereof are converted into a multi-page image MI. A chapter/section/item configuration of the book B is such that, as shown in FIG. 30. In FIG. 30, below ‘book name’, ‘chapters’ exist, and further below, ‘sections’ exist, and further below, ‘items’ exist.

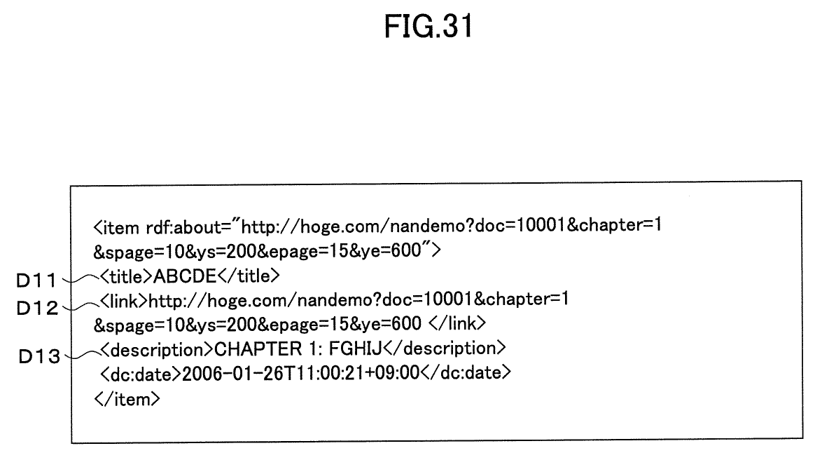

FIG. 31 shows an example of an RSS feed to be delivered. In a description D11 of a title tag, a book name is included. In a description D12 of a link tag, following to a document storage destination URL, a document number ‘doc’, a chapter number ‘chapter’, a starting page number ‘spage’, a starting y-coordinate ‘ys’, an ending page number ‘epage’, and an ending coordinate ‘ye’ are included. Further, in a description D13 of a description tag, a tile of ‘chapter’ is included. It is noted that the chapter number ‘chapter’ corresponds to the above-mentioned article number kiji. The starting page number ‘spage’, the starting coordinate ‘ys’, the ending page number ‘epage’ and the ending coordinate ‘ye’ correspond to the above-mentioned article area. It is noted that, different from a case where all the document elements included in one article are selected from one image, only the starting position (page and y-coordinate) and the ending position (page and y-coordinate) should be designated in this case.

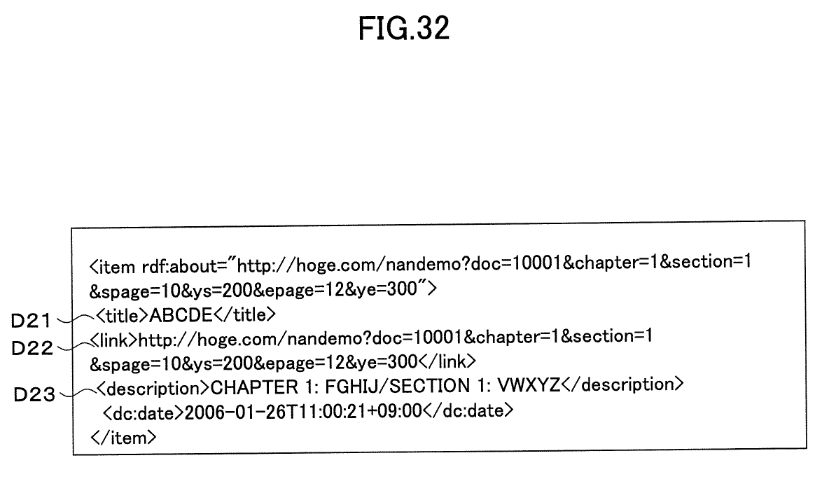

FIG. 32 shows another example of an RSS feed. ‘Section’ finer than ‘chapter’ is specified. That is, in a description D21 of a title tag, a book name is included. In a description D22 of a link tag, following to a document storage destination URL, a document number ‘doc’, a chapter number ‘chapter’, a section number ‘section’, a starting page number ‘spage’, a starting y-coordinate ‘ys’, an ending page number ‘epage’, an ending y-coordinate ‘ye’ are included. In a description D23 of a description tag, titles of the ‘chapter’ and ‘section’ are included. It is noted that, the chapter number ‘chapter’ and the section number ‘section’ correspond to the above-mentioned article number kiji, the starting page number ‘spage’, the starting y-coordinate ‘ys’, the ending page number ‘epage’ and the ending y-coordinates ‘ye’ correspond to the article area. Similarly, ‘item’ finer than ‘section’ may be further specified in the same way.

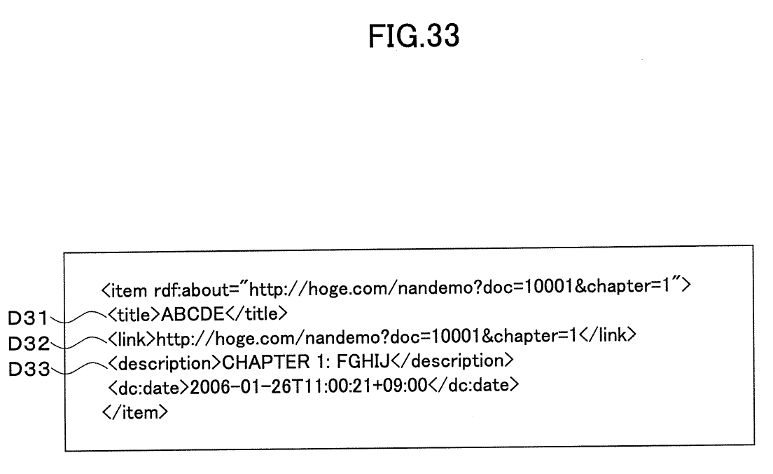

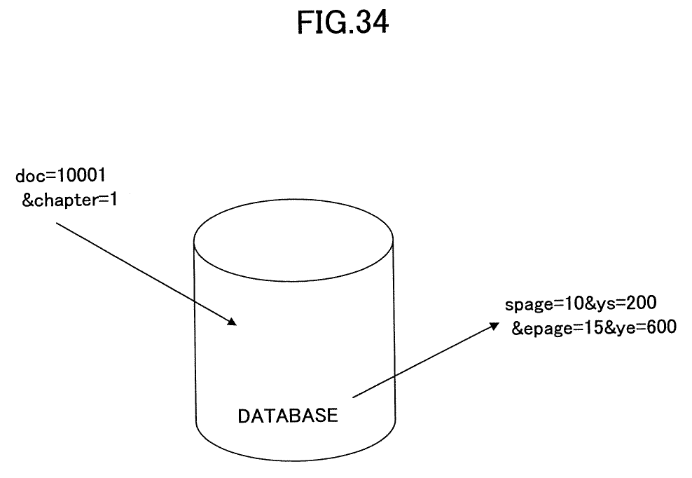

FIG. 33 shows another example of an RSS feed to be delivered. In this case, a starting page ‘spage’, a starting y-coordinate ‘ys’, an ending page ‘epage’ and an ending y-coordinate ‘ye’ are omitted, and only a chapter number ‘chapter’ is included. That is, in a description D31 of a title tag, a book name is included. In a description D32 of a link tag, following to a document storage destination URL, a document number ‘doc’, a chapter number ‘chapter’ are included. In a description D33 of a description tag, a title of ‘chapter’ is included. In this case, as a result of a corresponding relationship among the document number ‘doc’, the chapter number ‘chapter’, the starting page number ‘spage’, the starting y-coordinate ‘ys’, the ending page number ‘epage’ and the ending y-coordinates ‘ye’ being held in the database of the document management server 2 as shown in FIG. 34, it is possible to obtain the starting page number ‘spage’, the starting y-coordinate ‘ys’, the ending page number ‘epage’ and the enduing y-coordinate ‘ye’, i.e., a position of the ‘chapter, from the document number ‘doc’ and the chapter number ‘chapter’. The same way may be applied, also for ‘section’ or ‘item’.

<Processing for Reading>

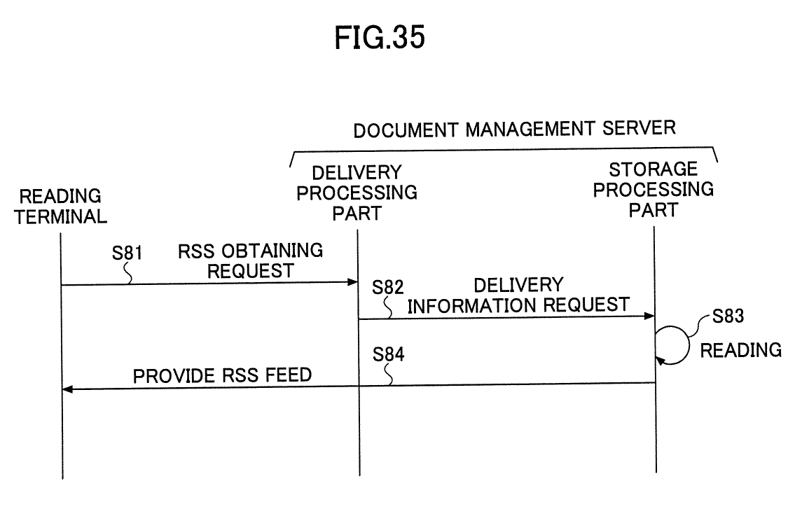

FIG. 35 shows a sequence diagram showing an example of processing for obtaining the meta data from the reading terminal 3R.

In FIG. 35, when the reading terminal 3R makes RSS obtaining request to the delivery processing part 23 of the document management server 2 (Step S81), the delivery processing part 23 requests the storage processing part 24 for providing delivery information (Step S82).

In response thereto, the storage processing part 24 reads the delivery information (Step S83), and provides an RSS feed including the meta data for each article (Step S84).

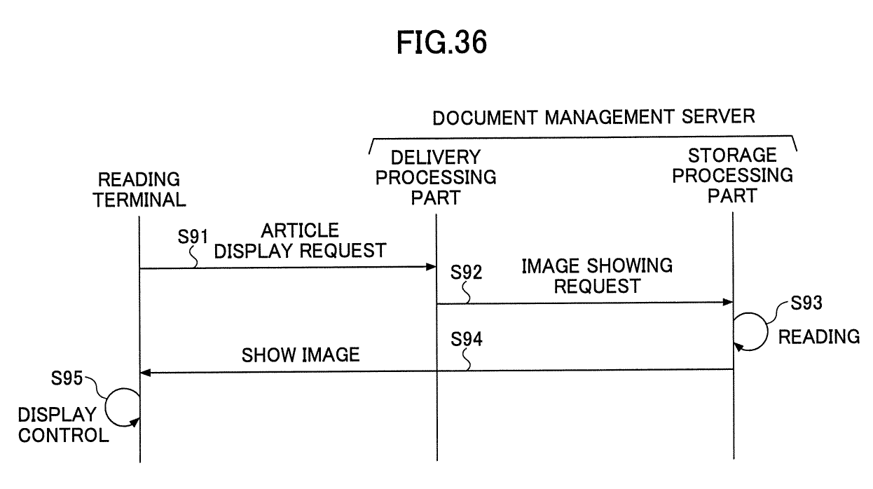

FIG. 36 shows a sequence diagram of an example of processing for display of the articles in the reading terminal 3R.

In FIG. 36, when article display request is made to the delivery processing part 23 of the document management server 2 from the reading terminal 3R (Step S91), the delivery processing part 24 requests the storage processing part 24 for providing an image (Step S92). It is noted that, in the article displays request, positional information of a target article in the document image is included or not included. When the positional information is not included, the positional information is obtained, as described above with reference to FIG. 28, from the document management server 2.

In response thereto, the storage processing part 24 reads corresponding image data (Step S93), and shows the image data to the reading terminal 3R. At this time, it possible that only a necessary scope may be extracted, with the use of the positional information for the article, and then, the extracted scope may be shown. It is noted that, it is also possible that, previously, image data is extracted for each article, is then stored, and, in response to a request, the same may be shown according to the request. Further, when the reading terminal 3R has not positional information of a target article, the storage processing part 24 may deliver the corresponding positional information to the reading terminal 3R together.

The reading terminal 3R receiving the image data then displays the corresponding image with carrying out display control based on the positional information (Step S95).

An RSS feed or such delivered to the reading terminal 3R has a small data size, and an image is delivered to the reading terminal only for an article for which display is actually required by a user. Accordingly, it is possible to minimize a necessary storage area in the reading terminal, and a necessary network load.

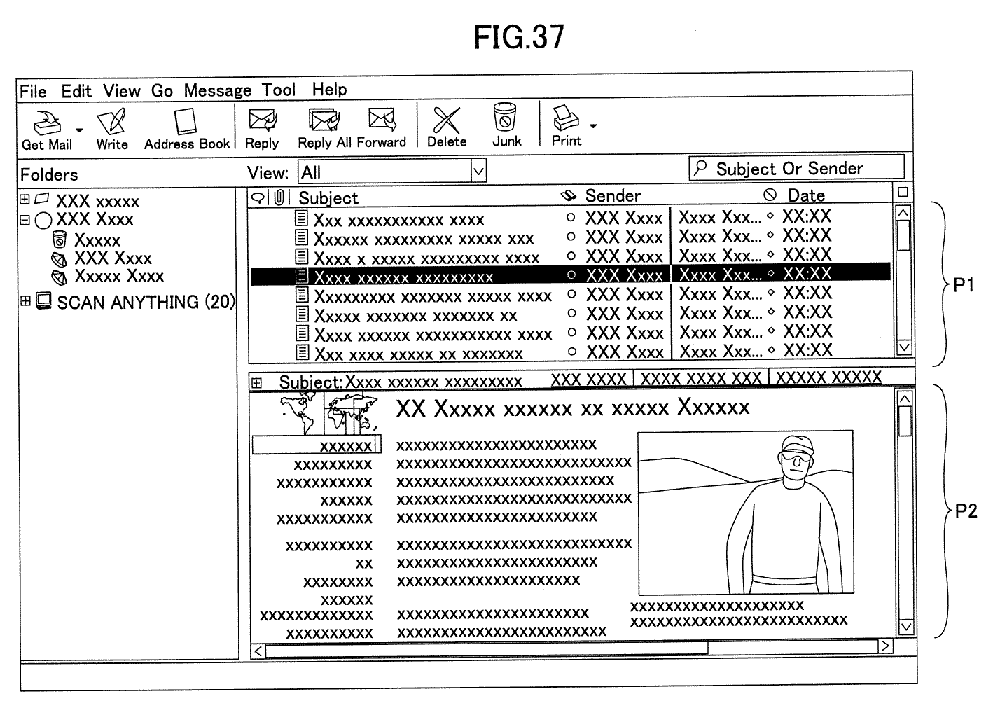

FIG. 37 shows an example of a reading page, in one article in one image, for when no positional information is used. FIG. 37 shows a state in which, in pane P1, articles based on an RSS feed delivered is displayed as a list, and, thereamong, an image of an article is selected by a user, which is then displayed in a pane P2.

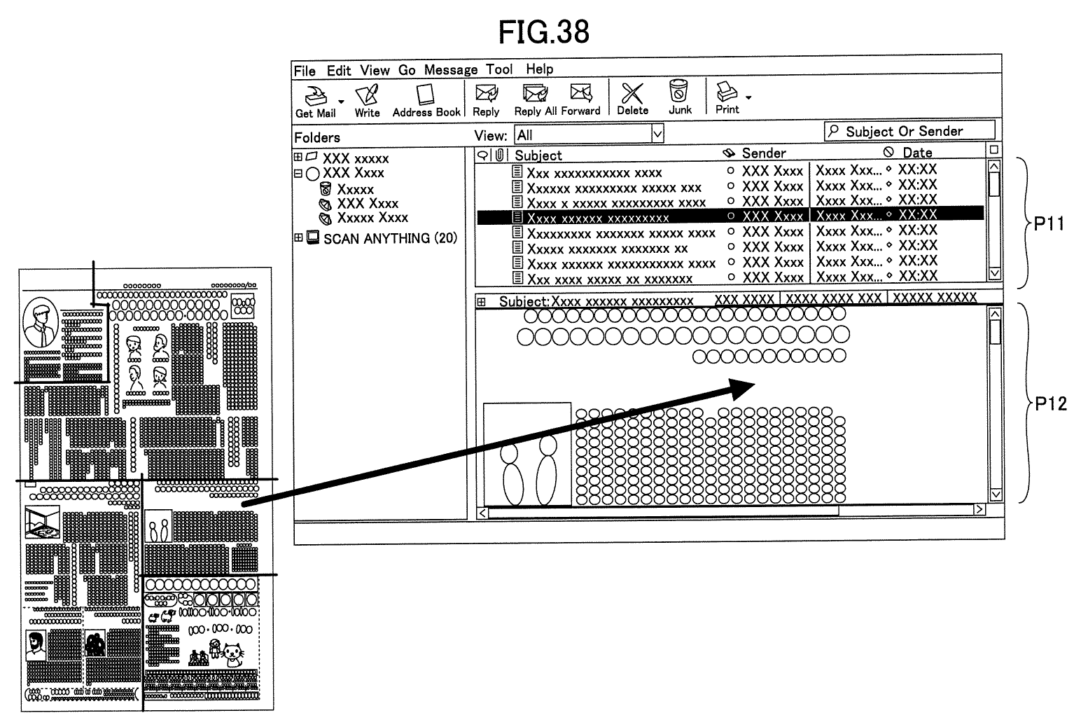

FIG. 38 shows an example of a reading page for when positional control is provided for a plurality of articles per one image. The same as the above-mentioned example of FIG. 37, in a pane P11, articles based on an RSS feed delivered are displayed as a list, and an image of an article selected by a user thereamong is displayed in a pane P12. In this example, based on positional information of an article, detecting a position corresponding to a top of the article image to be displayed in the pane P12 is carried out. Also for the above-mentioned multi-page image, a position corresponding to a top of a ‘chapter’, a ‘section or an ‘item’, designated, is detected in the same way. Thereby, it is easier to obtain a desired article. It is noted that, when it is not possible to divide a target article in a rectangle, and thus, it is not possible to properly detect a position corresponding to a top of the target article, it is possible to mask (i.e., to apply such a display manner that saturation is lowered or blurring is carried out, or such) adjacent articles, instead.

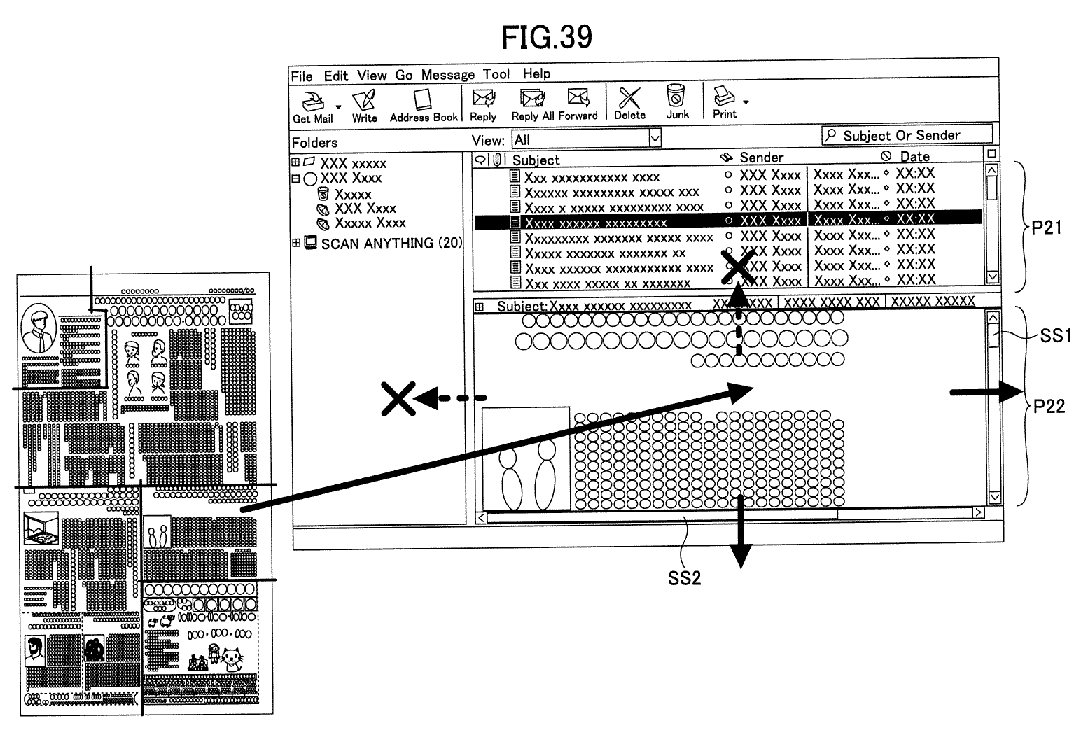

FIG. 39 shows an example of scrolling control. The same as the above-mentioned example of FIG. 38, in a pane P21, articles based on an RSS feed delivered are displayed as a list, and an image of an article selected by a user therefrom is displayed in a pane P22. In this example, based on positional information of an article, such control is made that moving to adjacent articles, which control is otherwise allowed by means of scrolling sliders SS1, SS2 for moving vertically or horizontally on article images displayed in the pane P22, is made not allowed. That is, in FIG. 39, scrolling downward or rightward in which target articles continue is allowed (in a case of the above-mentioned multi-page image, pages are displayed successively, page by page, in sequence, in response to scrolling downward when a display target exists in a plurality of pages), while, scrolling upward or leftward is not allowed. This manner may be applied to a system in which reading of an article is charged, or such. It is noted that, when a target article cannot be divided in a rectangle, and thus, a part of an adjacent article is inevitably shown with scrolling, the adjacent article may be masked.

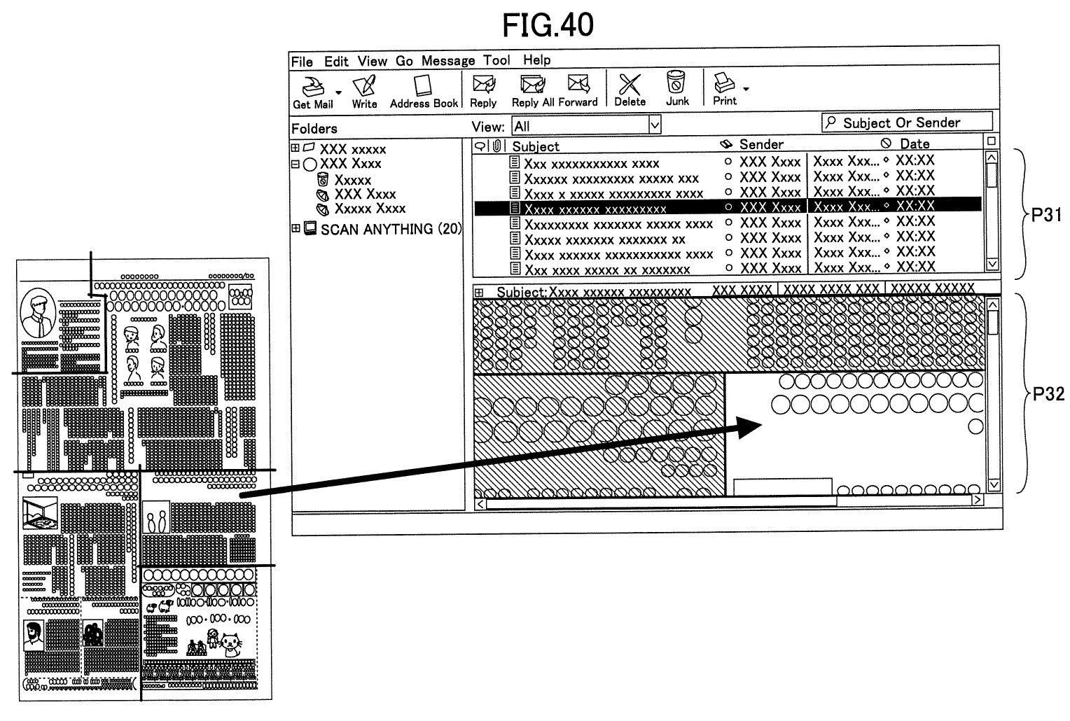

FIG. 40 shows an example in which display control is carried out for articles other than a target article. In this example, the same as the above-mentioned example of FIG. 38, in a pane P31, articles based on an RSS feed delivered are displayed as a list, and an image of an article selected by a user therefrom is displayed in a pane P32. In this example, although scrolling of article images displayed is allowed freely, articles other than the target article are masked. As a level of the masking, various modes may be applied, i.e., from such a level in which merely it is indicated that the articles are not included in the target article, to such a level that the article contents cannot be read at all. In particular, for the above-mentioned application to the system in which reading of an article is charged, it is necessary to use the level in which the articles other than the target article cannot be read at all.

The preferable embodiments of the present invention have been described above. There, the specific examples have been used to describe the present invention. However, the present invention is not limited to the above-described embodiments, and variations and modifications may be made without departing from the basic concept of the present invention claimed below.

The present application is based on Japanese Priority Applications Nos. 2006-113288 and 2007-96755, filed on Apr. 17, 2006 and Apr. 2, 2007, respectively, the entire contents of which are hereby incorporated herein by reference.

What is claimed is

1. An image reading system comprising:
a part obtaining image data created from reading a document by means of a scanning function;
a part creating positional information indicating an area of a particular article included in the image data;
a part creating meta data including bibliographic information of the article, information identifying the article and the positional information;
a part storing the thus-created meta data;
a part delivering to a terminal at least the bibliographic information and the information identifying the article;
a part delivering to the terminal the image data of the article in response to a request; and
a part carrying out display control of the article image based on the positional information when the meta data has the positional information of the article, while, when the meta data does not have the positional information of the article, carrying out display control of the article image based on the positional information obtained from the information identifying the article.

2. The image reading system as claimed in claim 1, wherein:
said image data to be delivered to the terminal comprises the entirety of the image data including the article required.

3. The image reading system as claimed in claim 1, wherein:
said image data to be delivered to the terminal comprises an area of the article extracted from the image data based on the positional information of the article requested.

4. The image reading system as claimed in any one of claims 1-3, wherein:
the creation of the positional information is carried out based on a position of an area including document elements, obtained from grouping the document elements which are included in the article, from among those obtained from decomposing an image included in the image data.

5. The image reading system as claimed in claim 4, wherein:
said grouping comprises selecting of the document elements.

6. The image reading system as claimed in claim 5, wherein:
said selecting of the document elements is carried out by means of operation of toggling between designation and cancellation.

7. The image reading system as claimed in claim 5, wherein:
said selecting of the document elements is carried out by means of setting a rectangular area, and selecting the document elements included therein.

8. The image reading system as claimed in claim 5, wherein:
said selecting of the document elements is carried out by means of setting a line segment, and selecting the document elements crossed by said line segment.

9. The image reading system as claimed in claim 4, wherein:
said grouping is carried out from designating of the article area, carried out by a user.

10. The image reading system as claimed in claim 1, wherein:
said display control comprises detecting a position corresponding to a top of the article image.

11. The image reading system as claimed in claim 1, wherein:
said display control comprises limiting a range of strolling in the target article area.

12. The image reading system as claimed in claim 1, wherein:
said display control comprises masking articles other than the target article.

13. A document management server comprising:
a part obtaining image data created from reading a document by means of a scanning function;
a part creating positional information indicating an area of a particular article included in the image data;
a part creating meta data including bibliographic information of the article, information identifying the article and the positional information;
a part storing the thus-created meta data;
a part delivering to a terminal at least the bibliographic information and the information identifying the article; and
a part delivering to the terminal the image data of the article in response to a request.

14. The document management server as claimed in claim 13, wherein:
said image data to be delivered to the terminal comprises the entirety of the image data including the article required.

15. The document management server as claimed in claim 13, wherein:
said image data to be delivered to the terminal comprises the area of the article extracted from the image data based on the positional information of the article requested.

16. The document management server as claimed in any one of claims 13-15, wherein:
the creation of the positional information is carried out based on a position of an area including document elements, obtained from grouping the document elements which are included in the article, from among those obtained from decomposing an image included in the image data.

17. The document management server as claimed in claim 16, wherein:
said grouping comprises selecting of the document elements.

18. The document management server as claimed in claim 16, wherein:
said grouping is carried out from designating of the article area, carried out by a user.

19. An image reading control method comprising:
the step of obtaining image data created from reading a document by means of a scanning function;
the step of creating positional information indicating an area of a particular article included in the image data;
the step of creating meta data including bibliographic information of the article, information identifying the article and the positional information;
the step of storing the thus-created meta data;
the step of delivering to a terminal at least the bibliographic information and the information identifying the article;
the step of delivering to the terminal the image data of the article in response to a request; and
the step of carrying out display control of the article image based on the positional information when the meta data has the positional information of the article, while, when the meta data does not have the positional information of the article, carrying out display control of the article image based on the positional information obtained from the information identifying the article.

20. An image reading control program comprising instructions for causing a computer to act as:
means for obtaining image data created from reading a document by means of a scanning function;
means for creating positional information indicating an area of a particular article included in the image data;
means for creating meta data including bibliographic information of the article, information identifying the article and the positional information;
means for storing the thus-created meta data;
means for delivering to a terminal at least the bibliographic information and the information identifying the article;
means for delivering to the terminal the image data of the article in response to a request; and
means for carrying out display control of the article image based on the positional information when the meta data has the positional information of the article, while, when the meta data does not have the positional information of the article, carrying out display control of the article image based on the positional information obtained from the information identifying the article.
